(19) United States
(12) Patent Application Publication
(10) Pub. No.: US 2023/0293308 A1
(43) Pub. Date: Sep. 21, 2023

(54) SYSTEM FOR BALANCING A HIP DURING A HIP ARTHROPLASTY

(71) Applicant: Arthrology Consulting, LLC, Palo Alto, CA (US)

(72) Inventor: Derek Amanatullah, Palo Alto, CA (US)

(21) Appl. No.: 18/123,177

(22) Filed: Mar. 17, 2023

Related U.S. Application Data
(60) Provisional application No. 63/321,013, filed on Mar. 17, 2022.

(51) Int. Cl. A61F 2/36

(52) U.S. Cl. CPC A61F 2/3609; A61F 2002/3611; A61F 2002/365

(57) ABSTRACT

Once variation of a system includes: a femoral head including a shank configured to seat on a neck of a femoral stem component installed on a femur, a spherical shell arranged over the shank, force sensors configured to output force data representing forces acting on the spherical shell, and inertial sensors configured to output orientation data representing orientations of the femoral head; a reference module configured to couple to a pelvis of a patient and output reference orientation data representing orientations of the pelvis; and a controller configured to access the force data, the orientation data, and the reference orientation data, calculate orientations of the femoral head relative the pelvis, and based on the orientations of the femoral head and the force data, calculate a force versus angular orientation curve representing forces exerted on the femoral head by the pelvis over a range of motion of the femur.

### CROSS-REFERENCE TO RELATED APPLICATIONS

This application claims priority to U.S. Provisional Patent Application No. 63/321,013, filed on 17 Mar. 2022, which is incorporated in its entirety by this reference.

### TECHNICAL FIELD

This invention relates generally to the field of arthroplasty and more specifically to a new and useful system for balancing a hip during a hip arthroplasty in the field of arthroplasty.

### BRIEF DESCRIPTION OF THE FIGURES

FIG. 1 is a flowchart representation of a system;

FIG. 2 is a flowchart representation of a system;

FIG. 3 is a graphical representation of one variation of the system;

FIG. 4 is a graphical representation of one variation of the system;

FIG. 5 is a graphical representation of one variation of the system;

FIG. 6 is a graphical representation of one variation of the system;

FIG. 7 is a graphical representation of one variation of the system;

FIG. 8 is a graphical representation of one variation of the system;

FIG. 9 is a graphical representation of one variation of the system;

FIG. 10 is a graphical representation of one variation of the system;

FIG. 11 is a graphical representation of one variation of the system; and

FIGS. 12A, 12B, and 12C are graphical representations of additional variations of the system.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. System

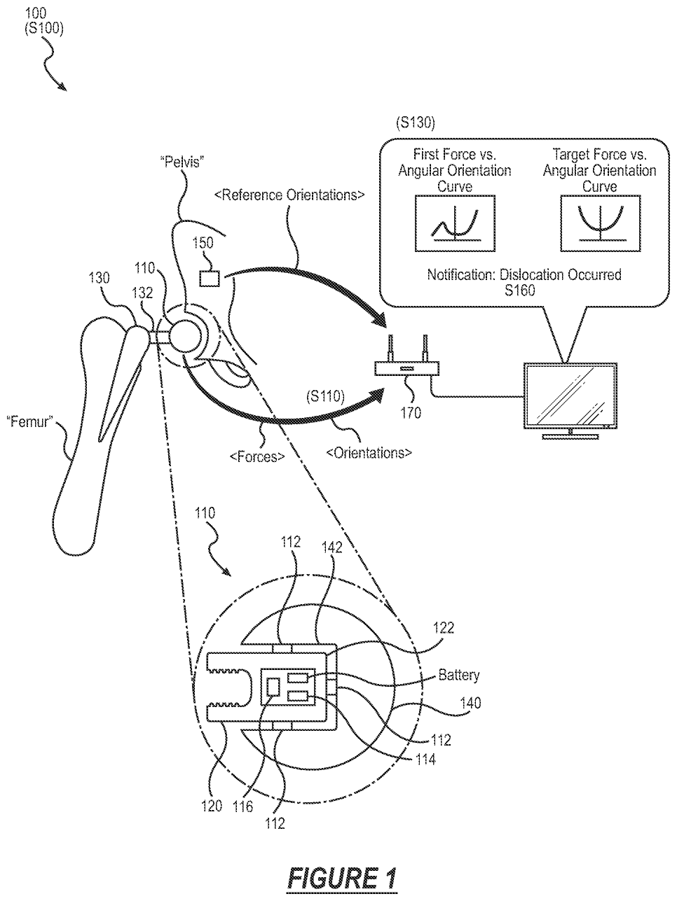

As shown in FIG. 1, a system 100 for balancing soft tissue tensions around a hip during a partial or total hip arthroplasty includes: an instrumented femoral head 110; a reference module 150 (also referred to as reference module 150); and a controller 170.

The instrumented femoral head 110 includes: a shank 120 configured to seat on a proximal end of a neck 132 of a femoral stem component 130 installed on a proximal end of a femur; a spherical shell 140 arranged over the shank 120; a force sensor 112 coupled to the shank 120 and the spherical shell 140 and configured to output a force signal representing longitudinal forces applied to the spherical shell 140 parallel to an axis of the neck 132 of the femoral stem component 130; and a first orientation sensor 118 configured to output a first orientation signal representing orientations of the spherical shell 140.

The reference module 150: is configured to couple to a pelvis; and includes a second orientation sensor configured to output a reference orientation signal representing orientations of the reference module 150.

The controller 170 is configured to: access the force signal, the first orientation signal, and the reference orientation signal following insertion of the instrumented femoral head no into the pelvis during the hip arthroplasty; interpret a sequence of relative orientations of the instrumented femoral head no relative to the pelvis based on the first orientation signal and the reference orientation signal; interpret a relationship between orientation of the instrumented femoral head 110 and longitudinal force on the instrumented femoral head no based on the force signal and the sequence of relative orientations; interpret inelastic deformation of soft tissue around the pelvis based on the relationship; and select a geometry of an artificial femoral head predicted to reduce inelastic deformation of soft tissue around the pelvis.

### 1.1 First Variation

As shown in FIG. 1, one variation of the system 100 includes an instrumented femoral head 110 including: a shank 120 configured to seat on a proximal end of a neck 132 of a femoral stem component 130 installed on a proximal end of a femur; a spherical shell 140 arranged over the shank 120; a set of force sensors 112 configured to output a first series of force data representing forces acting on the spherical shell 14o; and a set of inertial sensors 114 configured to output a second series of orientation data representing orientations of the instrumented femoral head 110.

This variation of the system further includes a reference module 150 configured to: couple to a pelvis of a patient; and output a third series of reference orientation data representing orientations of the pelvis.

This variation of the system 100 further includes a controller 170 configured to: access the first series of force data, the second series of orientation data, and the third series of reference orientation data; calculate a first sequence of orientations of the instrumented femoral head 110 relative the pelvis based on the second series of orientation data and the third series of reference orientation data; and, based on the first sequence of orientations of the instrumented femoral head 110 and the first series of force data, calculate a first force versus angular orientation curve representing forces exerted on the instrumented femoral head 110 by an acetabulum in the pelvis over a first range of motion of the femur.

### 1.2 Second Variation

As shown in FIG. 1, one variation of the system 100 includes: a shank 120 configured to seat on a proximal end of a neck 132 of a femoral stem component 130 installed on a proximal end of a femur and defining an external support surface 122; a spherical shell 140 arranged over the shank 120 and defining an internal contact surface 142 facing the external support surface 122 of the shank 120; a set of force sensors 112 interposed between the external support surface 122 of the shank 120 and the internal contract surface 142 of the spherical shell 140 and configured to locate the spherical shell 140 over the shank 120, communicate forces from the spherical shell 140 into the shank 120, and output a first series of force data representing forces acting on the spherical shell 140; a set of inertial sensors 114 arranged in the shank 120 and configured to output a second series of orientation data representing orientations of the instrumented femoral head no; and communication module 116 arranged in the shank 120 and configured to transmit the first series of force data, the second series of orientation data, and the third series of reference orientation data to a controller 170.

### 2. Method

As shown in FIG. 1, a method S100 includes, at a first time (e.g., after the first balancing routine): accessing a first series of force data from a set of force sensors 112, a second series of orientation data from a set of inertial sensors, and the third series of reference orientation data from a reference module 150 in Block Silo, the first series of force data representing forces acting on an instrumented femoral head 110 installed on a proximal end of a femoral stem component 130 attached to a femur of a patient, the second series of orientation data representing orientations of the instrumented femoral head no, and the third series of reference orientation representing reference orientations of a pelvis of the patient.

The method S100 further includes, at the first time: calculating a first sequence of orientations of the instrumented femoral head 110 relative the pelvis based on the second series of orientation data and the third series of reference orientation data in Block S120; based on the first sequence of orientations of the instrumented femoral head no and the first series of force data, calculating a first force versus angular orientation curve representing forces exerted on the instrumented femoral head 110 by an acetabulum in the pelvis over a first range of motion of the femur in Block S130; based on the first force versus angular orientation curve, identifying an impingement event in Block S140, the impingement event representing impingement of the instrumented femoral head 110 by an acetabulum or by a soft tissue; predicting a target geometry of the femoral stem component 130 associated with a target force versus angular orientation curve in Block S150, the target force versus angular orientation curve representing forces exerted on the instrumented femoral head 110 by an acetabulum in the pelvis over a first range of motion of the femur without impingement; and generating a recommendation to adjust geometry of the femoral stem component 130 to match the target geometry in Block S160, the geometry of the femoral stem component 130 defining an angle of the neck 132 of the femoral stem component 130 and a length of the neck 132 of the femoral stem component 130.

### 2. Applications

Generally, the system includes a set of instrumented components: temporarily installable within a hip during a hip arthroplasty; configured to relative orientations of a patient's femur relative to the patient's pelvis; and to capture longitudinal force on a neck 132 of a femoral stem component 130 installed on the patient's femur, which corresponds to tension (e.g., stress, strain) on soft tissues around the hip.

The system further includes a controller 170 (e.g., a computing device, a software application execution on a computer system) that: aggregates position and force data from the instrumented components during a sequence of hip balancing routines during the hip arthroplasty; derives relationships between position of the femur on the pelvis and longitudinal force on the instrumented femoral head 110, which predicts tensions on soft tissues around the hip over its range of motion, based on data collected during these hip balancing routines; calculates an artificial femoral head geometry that balances these soft tissue tensions (i.e., avoids inelastic strain to avoid tissue damage and a patient perception of “tightness” while maintaining a minimum tension to avoid patient perception of “looseness”); and/or calculates a change in depth of an acetabular component in the patient's pelvis that balances these soft tissue tensions.

In particular, during a hip arthroplasty, a surgeon may: resect the proximal end of the patient's femur; install a femoral stem component 130 on the femur; install the reference module 150 on the patient's pelvis; install a first or nominal instrumented femoral head 110 on the femoral stem component 130; seat the instrumented femoral head 110 within the patient's acetabulum; and manipulate the hip through a range of motion while the reference module 150 and the instrumented femoral head 110 capture orientation and/or longitudinal force data during a first hip balancing routine. The controller 170 can then: aggregate position and force data from the reference module 150 and the instrumented femoral head 110 during this first hip balancing routine; characterize tension on soft tissues around the patient's hip as a function of position of the hip; and predict a geometry (e.g., size, position on the femoral stem component 130) of an alternate instrumented femoral head 110 that will yield more balanced tensions on these soft tissues (e.g., avoid both inelastic deformation of soft tissues and achieve sufficient minimum tensions on the hip) throughout the range of motion of the hip.

The surgeon may then: replace the instrumented femoral head 110 on the femoral stem component 130 with the alternate instrumented femoral head 110; ream the patient's acetabulum; install an acetabular component in the acetabulum; seat the alternative instrumented femoral head 110 within the patient's acetabulum; and manipulate the hip through a range of motion while the reference module 150 and the instrumented femoral head 110 capture orientation and/or longitudinal force data during a second hip balancing routine. The controller 170 can then: aggregate position and force data from the reference module 150 and the instrumented femoral head 110 during this first hip balancing routine; characterize tension on soft tissues around the patient's hip as a function of position of the hip; predict a change in position (e.g., depth position, rotation) of the acetabular component that will yield more balanced tensions on these soft tissues throughout the range of motion of the hip; and prompt the surgeon to implement this change.

Upon completing this change, the surgeon may close the patient to complete the hip arthroplasty. Accordingly, the patient may perceive balanced soft tissue tensions in her artificial hip joint over its range of motion following recovery from the surgery—that is, sufficient soft tissue tension to avoid a sense of “looseness” without excess tension resulting in pain or tissue damage.

Therefore, the system can include a kit of (e.g., one or more) disposable instrumented femoral heads of various sizes or geometries that cooperate with the reference module 150 to: validate positions and geometries of an artificial femoral head, a femoral stem component 130, and an acetabular component in the patient's hip during a hip arthroplasty; recreate preoperative tensions across soft tissues around the hip over its range of motion in the post-operative hip with installed artificial components; and avoid post-operative dislocation events over the range of motion of the hip.

### 3. Artificial Hip Components

Generally, an artificial hip system can include: an acetabular component; an artificial femoral stem component 13o; and an artificial femoral head. The acetabular component defines: a hemispherical external section configured to seat in a reamed acetabulum; and an internal section defining a joint socket. The artificial femoral stem component 130 defines: a distal end including a stem configured to insert into a bore in the femur; and a proximal end defining a neck 132 configured to locate an instrumented femoral head no offset from the axis of the femur (e.g., the mechanical axis or kinetic axis of the femur). The artificial femoral head: includes a spherical end configured to install on and that is retained by the proximal end of the neck 132 of the femoral stem component iso; and is configured to mate with and rotate within the acetabular component.

### 4. Instrumented Femoral Head

As described above, the instrumented femoral head no includes: a shank 120 configured to seat on a proximal end of a neck 132 of a femoral stem component 130 installed on a proximal end of a femur; a spherical shell 140 arranged over the shank 120; a force sensor 112 coupled to the shank 120 and the spherical shell 140 and configured to output a force signal representing longitudinal forces applied to the spherical shell 140 parallel to an axis of the neck 132 of the femoral stem component 130; and a first orientation sensor configured to output a first orientation signal representing orientations of the spherical shell 140. In particular, the instrumented femoral head no: is configured to install on a proximal end of a neck 132 of an artificial femoral stem component 130; is configured to pivot in both an acetabulum of a pelvis and in the joint socket defined by the acetabular component; and contains both a force sensor 112 and an orientation sensor.

In one implementation, the instrumented femoral head 110 includes: a base; a spherical shell 140; an orientation sensor; a longitudinal force sensor 112; a wireless communication module 116; and a battery. In this implementation, the shank 120 is configured to install on (and rotationally index to) the proximal end of the femoral stem component 130. The spherical shell 140 is arranged over and sealed against the base. The orientation sensor is arranged inside the spherical shell 140. The longitudinal force sensor 112 is configured to detect magnitudes of forces applied to the spherical shell 140 in a direction parallel to (e.g., coaxial with) the neck 132 of the acetabular component when the instrumented femoral head 110 is installed on the femoral stem component 130. The wireless communication module 116 is arranged in the spherical shell 140 and is configured to broadcast orientation and longitudinal force signals output by the orientation and longitudinal force sensors 112. In one implementation, the communication module 116 is arranged in the shank 120 and configured to transmit a series of force data (e.g., data representing forces acting on the spherical shell 140), a series of orientation data (e.g., data representing orientations of the instrumented femoral head no), and the third series of reference orientation data (e.g., data representing orientations of the pelvis) to the controller 170.

For example, the longitudinal sensor can include a strain gauge or pressure sensor arranged between the shank 120 and spherical shell 140; and the spherical shell 140 can be sealed around the shank 120 and exhibit limited (e.g., less than 0.005″) longitudinal movement on the shank 120 when a longitudinal force is applied to the instrumented femoral head 110. In one implementation, the set of force sensors 112 of the instrumented femoral head 110 can include a set of piezoelectric load cells supporting the spherical shell 140 on the shank 120. Therefore, the instrumented femoral head 110 can detect dynamic forces transferred to the force sensors 112 by the spherical shell 140.

Additionally, or alternatively, the orientation sensor can include an IMU—such as including a three-axis gyroscope, a three-axis accelerometer, and a compass—and configured to output a signal representing angular velocities and linear accelerations of the instrumented femoral head 110 in three degrees of freedom in a global reference frame (e.g., relative to gravity). In one implementation, the set of inertial sensors 114 of the instrumented femoral head no and the reference module 150 can each include a three-axis inertial measurement unit (IMU). Therefore, the instrumented femoral head no and the reference module 150 can both detect linear accelerations and rotational velocities along three axis.

Furthermore, the instrumented femoral head no can define a disposable surgical tool. Accordingly: the shank 120 can be formed in a medical-grade stainless steel; the spherical shell 140 can be formed in a medical-grade acetal or stainless steel; and the battery can be sized to power the orientation sensor, the longitudinal force sensor 112, and the wireless communication module 116 for a duration of a surgical operation (e.g., up to four hours for a hip arthroplasty).

### 4.1 Force Sensor Arrangement

In one implementation, the shank 120 of the instrumented femoral head 110 defines an external support surface 122 and the spherical shell 140 of the instrumented femoral head no defines an internal contact surface 142 facing the external support surface 122 of the shank 120. In this implementation, the set of force sensors 112 of the instrumented femoral head no can be interposed between the external contract surface of the shank 120 and the internal contract surface of the spherical shell 140 locating the spherical shell 140 over the shank 120 and can communicate forces exerted on the instrumented femoral head no by an acetabulum from the spherical shell 140 onto the shank 120. Therefore, the instrumented femoral head 110 can include a set of force sensors 112 that can detect forces exerted on the instrumented femoral head 110 by an acetabulum in the pelvis. Furthermore, the spherical shell 140 of the instrumented femoral head no can transfer forces to the set of force sensors 112 arranged on the shank 120.

In one implementation, the external support surface 122 of the shank 120 of the instrumented femoral head no includes a base and a side. For example, the external support surface 122 of the shank 120 can be shaped like a cylinder, a rectangular prism, or a cube. In this implementation, the set of force sensors 112 include a first force sensor 112 arranged on the base of the external support surface 122, a second force sensor 112 arranged on the side of the external support surface 122 and proximal a longitudinal axis of the shank 120, a third force sensor 112 arranged on the side of the external support surface 122 opposite the second force sensor 112 and proximal the longitudinal axis of the shank 120, a fourth force sensor 112 arranged on the side of the external support surface 122 and proximal a lateral axis of the shank 120, and a fifth force sensor 112 arranged on the side of the external support surface 122 opposite the fourth force sensor 112 and proximal the lateral axis of the shank 120. Therefore, the instrumented femoral head no can include five force sensors 112 arranged inside the instrumented femoral head no such that each of the five force sensors 112 can detect a directional component of the force acting on the instrumented femoral head 110 along one of three axes.

In one implementation, the external support surface 122 of the shank 120 of the instrumented femoral head 110 includes a semi-spherical external support surface 122. For example, a portion of the external support surface 122 of the shank 120 can be shaped like a hemisphere. In this implementation, the set of force sensors 112 of the instrumented femoral head 110 includes a first force sensor 112, a second force sensor 112, and a third force sensor 112 arranged on the semi-spherical external support surface 122 and angularly spaced about a parallel of the semi-spherical external support surface 122, the parallel interposed between a proximal pole of the semi-spherical external support surface 122 and an equator of the semi-spherical external support surface 122. Therefore, the instrumented femoral head 110 can include three force sensors 112 arranged inside the instrumented femoral head 110 such that the three force sensors 112 can accurately detect forces exerted on the instrumented femoral head 110 by an acetabulum.

### 4.2 Data Collection Activation

In one implementation, the instrumented femoral head 110 can include a transmitter. In this implementation, the instrumented femoral head 110 can, at a first time, identify a first temporal pattern of force fluctuations in the first series of force data, the first temporal pattern including a set of force peaks of within a force activation range and occurring within an activation time interval. Then, in response to detecting the first temporal pattern, the instrumented femoral head 110 can activate a transmitter and trigger transmission of the first series of force data and the second series of orientation data, via the transmitter, to the controller 170. At a second time, the instrumented femoral head 110 can identify a second temporal pattern of force fluctuations in the first series of force data, the second temporal pattern different from the first temporal pattern and, in response to detecting the second temporal pattern, cease transmission of the first series of force data and the second series of orientation data to the controller 170. Therefore, the instrumented femoral head 110 can receive operator input (e.g., input from the surgeon) such as taps or force increases exerted on the instrumented femoral head no by the operator. In particular, three taps on the instrumented femoral head 110 can trigger transmission of the force data and orientation data to the controller 170. In addition, the instrumented femoral head 110 can stop transmission of data in response to operator input. In particular, the instrumented femoral head 110 can stop transmission of the first series of force data and the second series of orientation data to the controller 170 in response to receiving a second temporal pattern of taps (e.g., force increases resulting from pressing/tapping a finger on the instrumented femoral head 110).

In one implementation, the instrumented femoral head no can include a transmitter and a receiver. In this implementation, the instrumented femoral head no can, at a first time: receive, via the receiver, an activation signal from the controller 170 and, in response to receiving the activation signal, trigger transmission of the first series of force data and the second series of orientation data to the controller 170 via the transmitter. In addition, the instrumented femoral head no also can, at the second time: receive, via the receiver, a deactivation signal from the controller 170 and, in response to receiving the deactivation signal, deactivate the transmitter to stop transmitting the first series of force data and the second series of orientation data. Therefore, the instrumented femoral head no can receive operator input as signals from the controller 170. In response to receiving a first operator input (e.g., activation signal) from the controller 170, the instrumented femoral head 110 can trigger transmission of the data to the controller 170 and, in response to receiving a second operator input (e.g., deactivation signal) from the controller 170, the instrumented femoral head no can stop transmission of data to the controller 170.

### 4.3 Spring-loaded Instrumented Femoral Head

In one implementation, the instrumented femoral head no includes: a longitudinal stage configured to couple the shank 120 to the spherical shell 140 and configured to permit the spherical shell 140 to translate longitudinally on the shank 120 within a range of longitudinal distances (e.g., parallel to the axis of the neck 132 of the acetabular component); and a spring configured to drive the spherical shell 140 to its maximum proximal position on the base.

For example, the base: can be configured to seat on and index to the proximal end of the neck 132 of the femoral stem component 130; and define a post extending longitudinally from the neck 132 of the femoral stem component 130. In this example, the spherical shell 140 can define a counterbore that mates with and runs over the post. The instrumented femoral head 110 can further include a spring element: arranged in the counterbore and/or around the post; and configured to drive the spherical shell 140 to a maximal proximal position on the shank 120 and neck 132 of the femoral stem component 130. For example, the spring element can include a coil spring, an air spring, or a compressible elastomer block.

Therefore, the spherical shell 140 can compress the spring element and retract on the shank 120 of the instrumented femoral head no, thereby shortening the effective length of the neck 132 of the femoral stem component 130 when a force is applied to the instrumented femoral head no (e.g., by the patient's acetabulum or the acetabular component) in a direction parallel to the neck 132 of the femoral stem component 130. Accordingly, in this implementation, the instrumented femoral head 110 can yield reduced strain on soft and hard tissues in a patient's hip when the acetabular component, the femoral stem component 130, and the instrumented femoral head no are installed in the patient and the patient's hip is rotated during a hip balancing routine as described below.

4.4 Multiple Instrumented femoral head Geometries

In one variation, the system includes a kit of instrumented femoral heads, each: defining a different geometry—such as a different spherical shell 140 radius or different lateral, anteroposterior, or longitudinal shank 120 offset—that yields different effective lengths and angles of the femoral stem component 130 when installed in a patient.

### 5. Reference Module

As described above, the reference module 150: is configured to couple to a pelvis; and includes a second orientation sensor configured to output a reference orientation signal representing orientations of the reference module 150. In particular, the reference module 150 can detect changes in position of a patient's pelvis in an inertial reference frame as the instrumented femoral head 110 detects changes in position of the spherical shell 140 in the inertial reference frame. Accordingly, the controller 170 can derive relative changes in the position of the instrumented femoral head 110—and therefore the patient's femur—onto the patient's pelvis during a hip balancing routine by aligning and subtracting orientations of the reference module 150 from concurrent orientations of the instrumented femoral head no (both in the inertial reference frame), derived from data captured by orientation sensors in the reference module 150 and the instrumented femoral head 110, respectively.

In one implementation, the reference module 150 is configured to couple to a patient's pelvis, such as via a surgical adhesive or a threaded fastener. The reference module 150 also includes an orientation sensor, such as an IMU (e.g., including a three-axis gyroscope, a three-axis accelerometer, and a compass) configured to output a signal representing angular velocities and linear accelerations of the reference module 150 in three degrees of freedom in a global reference frame (e.g., relative to gravity).

Furthermore, the reference module 150 can include: a wireless communication module 116 configured to broadcast orientation signals output by the orientation sensor; and a battery sized to power the orientation sensor and the wireless communication module 116 for a duration of a surgical operation (e.g., up to four hours for a hip arthroplasty).

### 6. Controller

The controller 170: can define a standalone computer system or a software application executing on a computing device; can aggregate data broadcast by the instrumented femoral head no and the reference module 150; and can execute methods and techniques described below to guide the surgeon toward balancing soft tissues in the hip based on these data received from the instrumented femoral head no and the reference module 150.

### 7. Surgical Operation

During a hip arthroplasty, a surgeon may: open the patient's soft tissue around her hip to expose the patient's acetabulum and femoral head; resect the femoral head; ream the proximal end of the femur; and install a femoral stem component 130 in the femur, such as in a position that the surgeon interprets as approximating the geometry of the patient's native femoral head.

The surgeon then: selects an instrumented femoral head 110—from a kit of instrumented femoral heads of different sizes—of a particular size that approximates the size of the patient's native femoral head; and activates the instrumented femoral head no and the reference module 150. For example, the surgeon may switch on the reference module 150, the instrumented femoral head no, and the controller 170. Accordingly, the reference module 150, the instrumented femoral head no, and the controller 170 can wirelessly pair and then synchronize their internal clocks.

The surgeon then installs the reference module 150 on the patient's pelvis, such as with an adhesive or by inserting a threaded fastener through the reference module 150 into an exposed region of the patient's pelvis. The surgeon also installs the instrumented femoral head no on the proximal end of the femoral stem component 130; and seats the instrumented femoral head no in the patent's native acetabulum.

### 7.1 First Balancing Routine Prior to Acetabular Resection

Once activated, the reference module 150 streams timestamped positions (e.g., accelerations and orientations in three axes) to the computing device. Similarly, once activated, the instrumented femoral head no streams timestamped positions and force values to the computing device.

Once the surgeon has located the instrumented femoral head 110 in the patient's acetabulum, the surgeon indicates a neutral position of the hip, such as through the controller 170 directly, via a voice command to the controller 170, or through a foot pedal coupled to the controller 170. The surgeon then moves the hip over its range of motion, such as by articulating the hip through a spiral motion or randomly sweeping the hip over a range of positions. Alternatively, the surgeon may sequentially: confirm flexion-extension motion via the computing device and move the hip over its flexion-extension range of motion; confirm abduction-adduction motion via the computing device and move the hip over its abduction-adduction range of motion; and confirm internal-external motion via the computing device and move the hip over its internal-external range of motion.

During this first hip balancing routine, the controller 170 can: store a timestamp of receipt of confirmation of the neutral position (and/or motion types) entered by the surgeon; and collect timestamped position and force value data broadcast by the reference module 150 and the instrumented femoral head 110.

7.2 Hip Motion v. Longitudinal Force

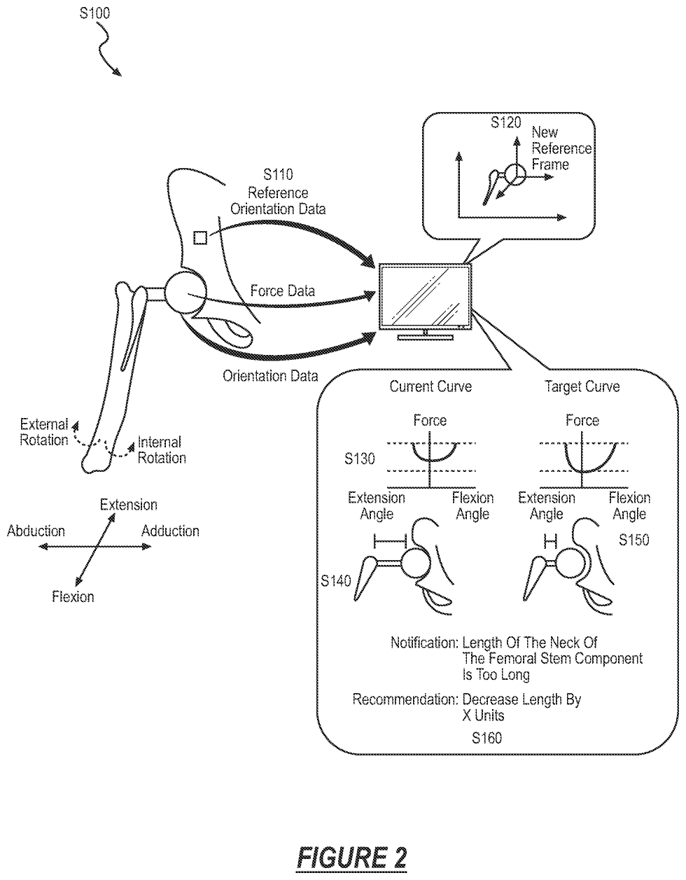

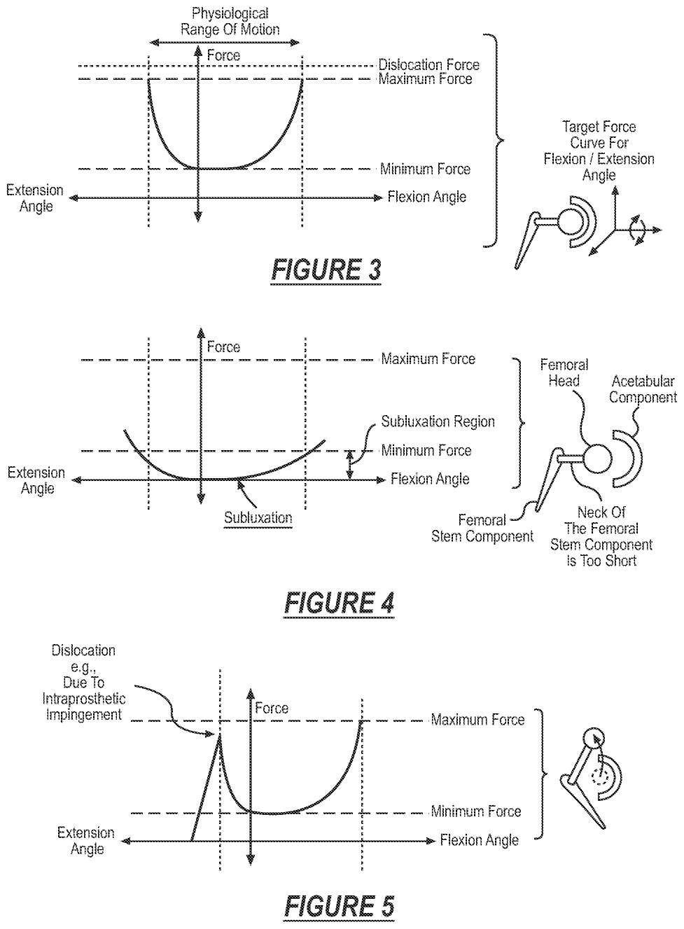

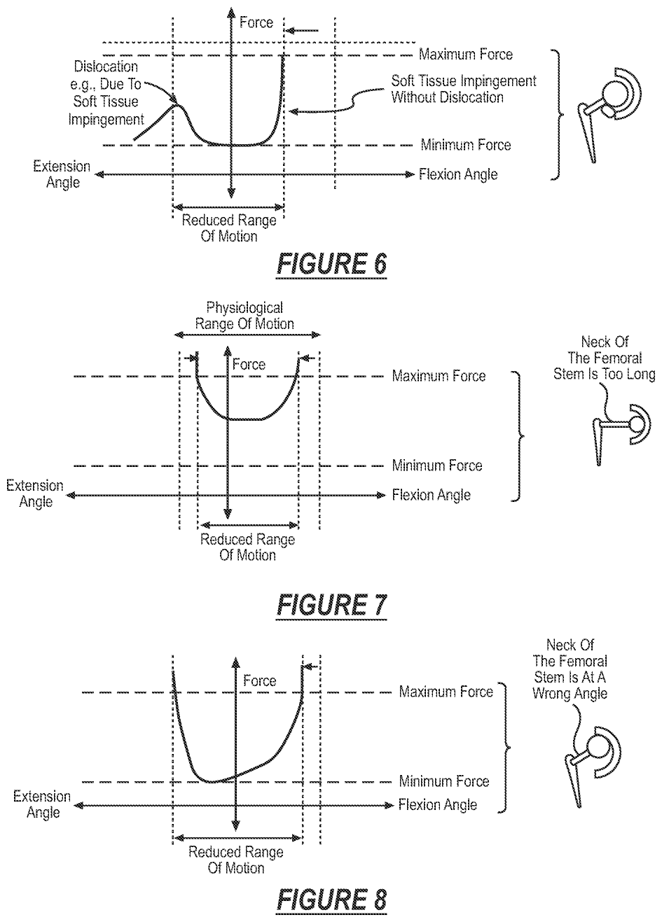

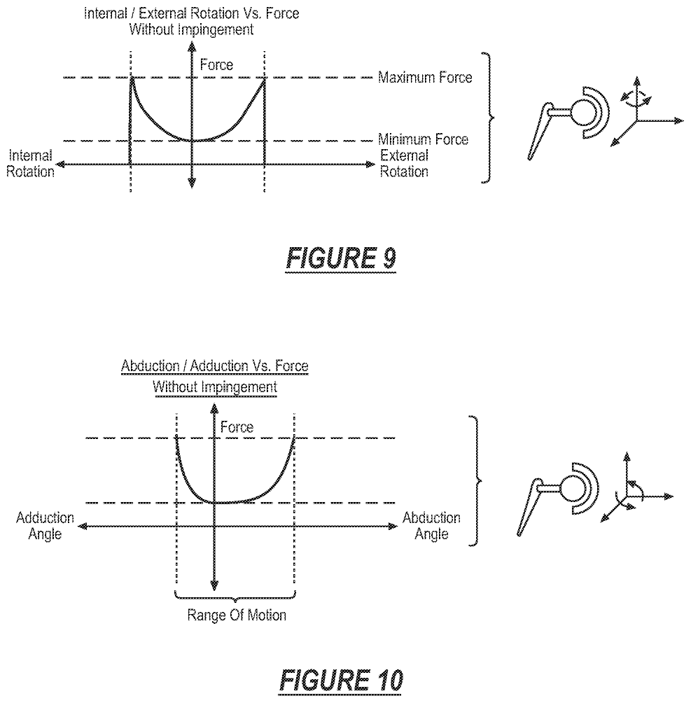

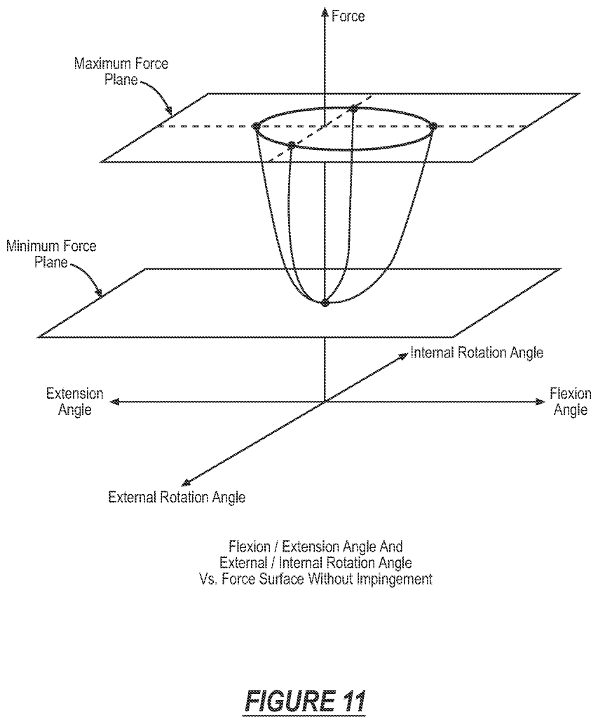

In one implementation shown in FIGS. 2-11, the controller 170: characterizes start positions of the reference module 150 and the instrumented femoral head 110 in orientation data received from the reference module 150 and the instrumented femoral head 110, respectively, at time of confirmation of the neutral position of the hip; characterizes a relative angular offset between the reference module 150 and the instrumented femoral head 110 in their corresponding start positions based on compass and gravity components detected in data received from the reference module 150 and the instrumented femoral head 110, respectively, at time of confirmation of the neutral position of the hip; and implements dead reckoning techniques (with gravity as a ground truth) to track the angular orientations of the reference module 150 and the instrumented femoral head 110 in the inertial reference frame based on position and orientation data received from the range of motion and the instrumented femoral head no. The controller 170 then subtracts angular orientations of the reference module 150 from concurrent angular orientations of the instrumented femoral head 110 to generate a timeseries of relative orientations of the instrumented femoral head no relative to the patient's pelvis during the first balancing routine.

The controller 170 can then: pair each relative orientation of the instrumented femoral head no with a concurrent force received from the instrumented femoral head no; and compile these relative orientation and force pairs into a surface (e.g., a 3D cone, a 4D hull) representing longitudinal force applied on the instrumented femoral head no as a function of flexion-extension, abduction-adduction, and/or internal-external motion of the hip, as shown in FIGS. 2-11.

Additionally or alternatively, the controller 170 can: compile relative orientation and force pairs—captured during flexion-extension motion of the hip—into a stress-position curve representing longitudinal forces on the instrumented femoral head no over the flexion-extension range of motion of the hip; compile relative orientation and force pairs—captured during abduction-adduction motion of the hip—into a stress-position curve representing longitudinal force on the instrumented femoral head no over the abduction-adduction range of motion of the hip; and/or compile relative orientation and force pairs—captured during internal-external motion of the hip—into a stress-position curve representing longitudinal force on the instrumented femoral head 110 over the internal-external range of motion of the hip.

7.2.1 Force versus Angular Orientation Curve

In one implementation, the controller 170 can: access the first series of force data from a set of force sensors 112, the second series of orientation data from a set of inertial sensors 114, and the third series of reference orientation data from a reference module 150 during flexion and extension motion of a hip of the patient. The controller 170 can then calculate a first force versus angular orientation curve representing forces exerted on the instrumented femoral head 110 by an acetabulum in the pelvis over a first range of motion of the femur by: calculating a first force versus angular orientation curve representing forces exerted on the instrumented femoral head 110 by the acetabulum over a flexion-extension range of motion of the femur. In this implementation, the controller 170 can also access this data and construct a force versus flexion angle and extension angle curve. Therefore, data collected by the instrumented femoral head 110 while the femur is undergoing flexion and/or extension motion can be used by the controller 170 to construct a force versus flexion angle and extension angle curve.

In one implementation, the controller 170 can: access a fourth series of force data from a set of force sensors 112, a fifth series of orientation data from a set of inertial sensors 114, and a sixth series of reference orientation data from a reference module 150 during abduction and adduction motion of the hip of the patient; calculate a second sequence of orientations of the instrumented femoral head 110 relative the pelvis based on the fifth series of orientation data and the sixth series of reference orientation data; based on the second sequence of orientations of the instrumented femoral head 110 and the fourth series of force data, calculate a second force versus angular orientation curve representing forces exerted on the instrumented femoral head 110 by an acetabulum in the pelvis over an abduction and adduction range of motion of the femur. Therefore, the instrumented femoral head no can collect data while femur is undergoing abduction and/or adduction motion. The controller 170 can then access this data and construct a force versus abduction angle and adduction angle curve.

In one implementation, the controller 170 can, access the first sequence of orientations of the instrumented femoral head no, each orientation in the first sequence of orientations including: a first vector component indicating orientation of the instrumented femoral head no parallel to a gravitational force; a second vector component indicating orientation of the instrumented femoral head 110 orthogonal to the first vector component; and a third vector component indicating orientation of the instrumented femoral head 110 orthogonal to the first vector component and the second vector component. In this implementation, the controller 170 can further: identify a first subset of orientations, in the first sequence of orientations, including second vector components within a narrow value range and first vector components and third vector components spanning a wide value range; isolate a first subset of forces in the first series of force data corresponding to the first subset of orientations; associate the first subset of orientations with the first range of motion for internal rotation and external rotation of the hip; and generate the first force versus angular orientation curve including a force versus internal rotation angle and external rotation angle curve based on the first subset of forces and the first subset of orientations. Therefore, the controller 170 can, based on the data collected by the instrumented femoral head no and the reference module 150, identify the motion (e.g., flexion, extension, abduction, adduction, internal rotation, external rotation, or a combination of the above) of the hip of the patient by detecting rotation of the femoral head about an axis. For example, if the femoral head rotates about an axis perpendicular to the gravity vector (e.g., while the patient is laying down on an operating table), the hip is undergoing external rotation or internal rotation. Once the motion is identified, the controller 170 can construct a two-dimensional graph plotting force versus angle of rotation. For example, the two-dimensional graph plotting force versus angle of internal rotation and angle of external rotation can include forces represented on the y-axis, internal rotation angles represented on the positive x-axis, and the external rotation angles represented on the negative x-axis.

In one implementation, the controller 170 can: identify a second subset of orientations, in the first sequence of orientations, including first vector components within the narrow value range and second vector components and third vector components spanning the wide value range; identify a second subset of force data in the first series of force data corresponding to the second subset of orientations; isolate a second subset of forces in the first series of force data corresponding to the second subset of orientations; associate the second subset of orientations with a second range of motion for abduction and adduction of the hip; and generate a force versus abduction and adduction curve based on the second subset of forces and the second subset of orientations. Therefore, the controller 170 can, based on the data collected by the instrumented femoral head no and the reference module iso, identify the motion (e.g., flexion, extension, abduction, adduction, internal rotation, external rotation, or a combination of the above) of the hip of the patient. For example, if the instrumented femoral head no rotates about an axis parallel to the gravity vector (e.g., while the patient is laying down on an operating table), the hip is undergoing external rotation or internal rotation.

### 7.2.2 Range of Motion and Force Curve

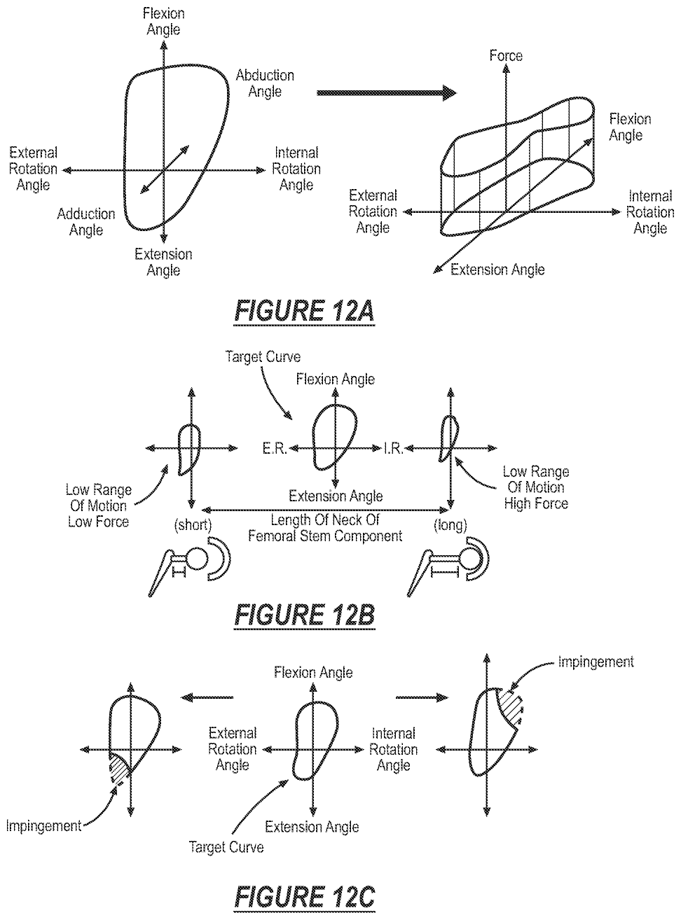

In one implementation shown in FIGS. 12A-12C, the controller 170 can: access the first series of force data, the second series of orientation data, and the third series of reference orientation data; calculate a first sequence of orientations of the instrumented femoral head 110 relative the pelvis based on the second series of orientation data and the third series of reference orientation data; and based on the first sequence of orientations of the instrumented femoral head no and the first series of force data, calculate a range of motion curve representing range of motion of the instrumented femoral head no and force exerted on the instrumented femoral head 110. For example, the range of motion curve can include flexion and extension angles of the hip plotted on a first axis, external rotation and internal rotation angles plotted on a second axis, abduction and adduction angles plotted on a third axis, and force indicated by a color gradient. In another example, the range of motion curve can include flexion and extension angles of the hip plotted on a first axis, external rotation and internal rotation angles of the hip plotted on a second axis, and forces exerted on the instrumented femoral head no plotted on a third axis. Generally, the controller and/or the surgeon can use the range of motion curve to detect impingement or to identify target orientation of the femoral stem component and the instrumented femoral head no relative to the acetabulum.

In one implementation, the controller 170 can generate a recommendation to adjust the length of the neck 132 of the femoral stem component 130 based on the range of motion curve. In particular, following an articulation of the hip through a range of motion by the surgeon, the controller 170 can calculate a range of motion curve, the range of motion curve representing a maximum range of motion of the hip without impingement. In response to identifying that the range of motion curve is smaller (e.g., having a smaller area enclosed by the curve) than a target range of motion curve (e.g., range of motion curve for an average healthy patient) while having a low average force, the controller 170 can detect subluxation of the instrumented femoral head 110 and generate a recommendation to increase the length of the neck 132 of the femoral stem component 130. In response to identifying that the range of motion curve is smaller than the target range of motion curve while having a high average force, the controller 170 can generate a recommendation to decrease the length of the neck 132 of the femoral stem component 130. In addition, the controller 170 can identify a target length of the neck 132 of the femoral stem component 130 based on the range of motion curve. For example, the controller 170 can calculate a similarity score for the range of motion curve and a target range of motion curve. In response to the similarity score exceeding a threshold similarity score, the controller 170 can generate a recommendation to maintain the current length of the neck 132 of the femoral stem component 130.

In one implementation, the controller 170 can identify impingement (e.g., impingement on the bone, impingement on the soft tissue) of the instrumented femoral head no based on the range of motion curve. In particular, the controller 170 can identify impingement of the instrumented femoral head no in response to identifying an insufficient the range of motion available to the hip and/or the instrumented femoral head no. For example, following an articulation of the hip through a range of motion by the surgeon, the controller 170 can calculate a range of motion curve, the range of motion curve representing the maximum range of motion of the hip without impingement. The controller 170 can then a identify an insufficient range of motion in a target region (e.g., flexion and internal rotation region) of the range of motion curve by, for example, calculating the area covered by the range of motion curve in the region and accessing the area covered by a target range of motion curve in that region. Then, in response to calculating that the area covered by the target range of motion curve in the region exceeds the area covered by the range of motion curve in the region by over a threshold percentage, the controller 170 can generate a notification indicating impingement of the instrumented femoral head no (e.g., due to orientation of the acetabular component deviating from a target orientation, due to the angle of the neck 132 of the femoral stem component 130 deviating from a target angle). Generally, when analyzing the range of motion curve to identify impingement, the controller 170 can first identify possible differences between the target range of motion curve and the range of motion curve in the flexion and internal rotation region and in the extension and external rotation region, as impingement may likely occur when the hip is simultaneously flexed and internally rotated or when the hip is simultaneously extended and externally rotated.

### 7.3 Ball Size Selection

Based on these data, the controller 170 can then predict changes in lateral, vertical, and/or anteroposterior position of the center of the instrumented femoral head 110 that is likely to yield: a target stress-position curve over a flexion-extension range of motion; a target stress-position curve over an abduction-adduction range of motion; a target stress-position curve over an internal-external range of motion; a target stress-position surface over complete three-dimensional range of motion; more than a minimum force at any hip position within the range of motion; and/or less than a maximum force at any hip position within the range of motion.

More specifically, based on the foregoing stress-position curves derived from data captured during the first hip balancing routine, the controller 170 can calculate a new position of the center of the instrumented femoral head 110 predicted to: maintain flexion-extension motion of the hip within an elastic range of the flexion-extension stress-position curve; maintain abduction-adduction motion of the hip within an elastic range of the abduction-adduction stress-position curve; and maintain internal-external motion of the hip within an elastic range of the internal-external stress-position curve.

The controller 170 can then: calculate a target femoral head geometry that fulfills this new position of the center of the instrumented femoral head 110, such as based on a known geometry of the instrumented femoral head 110; and identify a second instrumented femoral head 110—in the kit of instrumented femoral heads—that best approximates the target femoral head geometry.

7.3.1 Example: Shorten Effective Femoral stem component Neck Length

In one example, the controller 170 detects: high longitudinal force on the instrumented femoral head 110 in the neutral hip position; longitudinal forces that drop equally over extension-flexion and abduction-adduction ranges of motion; and longitudinal forces that remain consistent over the internal-external range of motion of the hip. More specifically, in this example, soft tissue around the hip may be under excess tension in the neutral position, as indicated by small changes in longitudinal force on the instrumented femoral head 110 resulting from large changes in position of the hip around the neutral position. Furthermore, in this example, the hip may be balanced at its greater extension-flexion and abduction-adduction motion limits, as indicated by linear changes in longitudinal force on the instrumented femoral head 110 resulting from corresponding changes in position of the hip near its extension-flexion and abduction-adduction motion limits. Accordingly, in this example, the controller 170 can predict that the effective length of the neck 132 of the femoral stem component 130 is too long, but that the angle and orientation of the femoral stem component 130 is accurate. The controller 170 can further select a second instrumented femoral head no of same spherical radius as the instrumented femoral head no, but with a deeper bore configured to seat further on the neck 132 of the femoral stem component 130 and thus reduce the effective length of the neck 132 of the femoral stem component 130.

Alternatively, in the foregoing example, the controller 170 can select a second instrumented femoral head no of smaller spherical radius than the instrumented femoral head 110, but a same bore depth as the instrumented femoral head no. In this example, replacement of the instrumented femoral head no with the second, smaller artificial femoral head moves the center of the second instrumented femoral head no closer to the axis of the femur and thus reduces the effective length of the neck 132 of the femoral stem component 130.

Yet alternatively, the controller 170 can select a shim geometry configured to relocate the instrumented femoral head no further down the neck 132 of the femoral stem component 130 when installed between the instrumented femoral head 110 and the femoral stem component 130.

### 7.3.2 Example: Shift Instrumented Femoral Head Center Rearward

In another example, the controller 170 detects: elastic strain on soft tissue at and around the neutral position; low force and nonlinear strain on soft tissue in extension; and high force and inelastic strain on soft tissue in flexion. In this example, the controller 170 can: predict that the effective neck 132 length and angle of the femoral stem component 130 are correct; and identify a need to offset the artificial femoral head in the anterior direction.

Accordingly, the controller 170 can prompt the surgeon to rotate the femoral stem component 130 in the femur to shift the proximal end of the neck 132 in the anterior direction. Alternatively, the controller 170 can select a second instrumented femoral head 110 defining an offset bore; and calculate an orientation of the second instrumented femoral head 110 on the femoral stem component 130 that shifts the effective center of the second instrumented femoral head 110 rearward. Yet alternatively, the controller 170 can calculate a shim geometry predicted to relocate the instrumented femoral head no rearward when installed between the instrumented femoral head 110 and the femoral stem component 130.

### 7.3.3 Example: Shift Instrumented Femoral Head Center Rearward

In another example, the controller 170 detects: low force and nonlinear strain on soft tissue at and around the neutral position; high force and inelastic strain on soft tissue in adduction; and low force and nonlinear strain on soft tissue in abduction. Accordingly, the controller 170 can prompt the surgeon to replace the femoral stem component 130 with a second femoral stem component 130 defining a steeper neck 132 angle to shift the instrumented femoral head 110—once installed on the proximal end of the neck 132—upwardly in the vertical direction. Alternatively, the controller 170 can: select a second instrumented femoral head 110 defining an offset bore; and calculate an orientation of the second instrumented femoral head 110—on the femoral stem component 130—that locates the center of the second instrumented femoral head no further offset vertically above the femur, thereby increasing the effective angle of the femoral stem component 130. Yet alternatively, the controller 170 can calculate a shim geometry configured to relocate the instrumented femoral head 110 accordingly when installed between the instrumented femoral head no and the femoral stem component 130.

### 7.3.4 Example: Shorten the Neck of the Femoral Stem Component

In one implementation, the controller 170 can detect a global minimum force of the first force versus angular orientation curve and, in response to the global minimum force exceeding a minimum force threshold: detect impingement of the instrumented femoral head no and the acetabulum resulting from a current length of the neck 132 of the femoral stem component 130 exceeding a target length; predict the target length of the neck 132 of the femoral stem component 130, the target length less than the current length of the neck 132 of the femoral stem component 130, based on a difference between the global minimum force and the minimum force threshold; generate a recommendation to shorten the current length of the neck 132 of the femoral stem component 130 according to the target length; and transmit the recommendation to the display for presentation to a surgeon. Therefore, the controller 170 can, based on certain features of the force versus angular orientation curve (e.g., the global minimum force of the first force versus angular orientation curve exceeding a threshold), detect that the geometry of the femoral stem component 130 results in an impingement of the femoral stem component 130. In particular, the controller 170 can determine that the neck 132 of the femoral stem component 130 exceeds a target length of the neck 132 of the femoral stem component 130, which can decrease the range of motion of the hip of the patient. Therefore, the controller 170 can generate a recommendation to reduce the length of the neck 132 of the femoral stem component 130 and transmit the recommendation to a display communicatively coupled to the controller 170 enabling the surgeon to view the recommendation.

### 7.3.5 Example: Identifying Subluxation of the Femoral Stem Component

In one implementation, the controller 170 can identify a characteristic of the force versus angular orientation curve, the characteristic including a global minimum force of the force versus angular orientation curve and, based on the global minimum force falling below a minimum force threshold, associate the characteristic with a subluxation of the femoral stem component 130 in the acetabulum of the pelvis. In this implementation, the controller 170 can further: generate a notification indicating the subluxation of the femoral stem component 130, in response to associating the first characteristic with a subluxation of the femoral stem component 130; generate a recommendation to increase a length of the neck 132 of the femoral stem component 130; and transmit the notification to a display communicatively coupled to the controller 170 and arranged in an operating theater. In another example, the controller 170 can: detect a global minimum force of the first force versus angular orientation curve and in response to the global minimum force falling below a minimum force threshold; detect subluxation of the femoral stem component 130 in the acetabulum of the pelvis; generate a recommendation to increase a length of the neck 132 of the femoral stem component 130; and render the recommendation on a display arranged proximal the patient. Therefore, the controller 170 can, based on certain features (e.g., characteristics) of the force versus angular orientation curve, identify that the geometry of the femoral stem component 130 causes a subluxation of the femoral stem component 130. In particular, the controller 170 can determine that the neck 132 femoral stem component 130 falls below a target length of the neck 132 femoral stem component 130, which leads to the subluxation of the hip of the patient. Therefore, the controller 170 can generate a recommendation to increase the length of the neck 132 of the femoral stem component 130 and transmit the recommendation to a display enabling the surgeon to view the recommendation.

### 7.3.6 Example: Identifying Dislocation of the Femoral Stem Component

In one implementation, the controller 170 is configured to define the first range of motion of the femur, which includes a set of angular orientations accessible to the femur, each angular orientation in the set of angular orientations associated with a force falling below a maximum threshold force. In this implementation, the controller 170 can identify a first characteristic of the force versus angular orientation curve, the first characteristic including a discontinuity in the force versus angular orientation curve, the discontinuity occurring within the range of motion of the femur and identify a second characteristic of the force versus angular orientation curve, the second characteristic including a maximum force at the discontinuity, the maximum force at the discontinuity falling below the maximum threshold force. Then, based on matching the first characteristic and the second characteristic to a template curve characteristic stored in a library and associated with a dislocation event, the controller 170 can: predict dislocation of the femoral stem component 130 from the acetabulum; generate a notification indicating predicted dislocation of the femoral stem component 130 from the acetabulum; and render the notification on a display proximal the patient. Therefore, the controller 170 can, based on certain characteristics of the force versus angular orientation curve, identify a dislocation of the femoral stem component 130 (e.g., due to intraprosthetic impingement). In particular, the controller 170 can access a library of known characteristics of possible force versus angular orientation curves and, based on the identified characteristics of the force versus angular orientation curve matching certain known characteristics, determine that the force versus angular orientation curve is indicative of a dislocation of the femoral stem component 130 due to intraprosthetic impingement. To deliver this information to the surgeon, the controller 170 can generate a notification indicating dislocation of the femoral stem component 130.

### 7.3.7 Example: Identifying Impingement of a Soft Tissue

In one implementation, the controller 170 can define the first range of motion of the femur as the range of motion that includes a set of angular orientations accessible to the femur, each angular orientation in the set of angular orientations associated with a force below a maximum threshold force. Therefore, the first range of motion of the femur is associated with a range of forces where the maximum threshold force is an upper bound on the range of forces. In this implementation, the controller 170 can identify a first characteristic of the force versus angular orientation curve, the first characteristic including a peak in the force versus angular orientation curve and identify a second characteristic of the force versus angular orientation curve, the second characteristic including a maximum force associated with the peak, the maximum force falling below the maximum threshold force. Then, based on matching the first characteristic and the second characteristic to a soft tissue impingement event in a library of known force versus angular orientation curve characteristics, the controller 170 can associate the first characteristic and the second characteristic with an impingement of the femoral stem component 130 and a soft tissue, and generate a notification indicating the soft tissue impingement event. Therefore, the controller 170 can, based on certain features (e.g., characteristics) of the force versus angular orientation curve, identify an impingement of the femoral stem component 130 and a soft tissue, which does not lead to a dislocation. In particular, the features of the force versus angular orientation curve can include a peak in force across a range of angular orientations. In particular, the controller 170 can access a library of known characteristics of possible force versus angular orientation curves and, based on the peak in the force versus angular orientation curve being associated with a soft tissue impingement of the femoral stem component 130, determine that the force versus angular orientation curve is indicative of a soft tissue impingement of the femoral stem component 130. To deliver this information to the surgeon, the controller 170 can generate a notification indicating dislocation of the femoral stem component 130. To deliver this information to the surgeon, the controller 170 can generate a notification indicating dislocation of the femoral stem component 130.

### 7.3.8 Example: Adjusting Angle of the Neck of the Femoral Stem Component

In one implementation, the controller 170 can detect a first characteristic of the force versus angular orientation curve, the first characteristic including an asymmetric profile of the force versus angular orientation curve across the first range of motion. The controller 170 can further: associate the first characteristic with an angle of the neck 132 of the femoral stem component 130 deviating from a target angle; generate a target force versus angular orientation curve; based on the target force versus angular orientation curve, calculate the target angle of the neck 132 of the femoral stem component 130 predicted to reduce asymmetry of the first force versus angular orientation curve; and generate a recommendation to adjust the angle of the neck 132 of the femoral stem component 130 to the target angle. Therefore, the controller 170 can, based on the general shape of the force versus angular orientation curve, detect that the geometry of the femoral stem component 130 causes a decreased range of motion of the femoral stem component 130 and an imbalance of pressure acting on the femoral head across the range of motion. In particular, the controller 170 can determine that the angle of the neck 132 femoral stem deviates from a target angle of the neck 132 femoral stem component 130, which results in the uneven distribution of forces on the femoral stem component 130 across a range of motion. Therefore, the controller 170 can generate a recommendation to alter the angle of the neck 132 of the femoral stem component 130.

### 7.3.9 Example: Identifying Target Geometry of the Femoral Stem Component

In one implementation, the controller 170 can: access a target force versus angular orientation curve; calculate a similarity score for the first force versus angular orientation curve and the target force versus angular orientation curve; and, based on the similarity score exceeding a threshold similarity score generate a notification confirming an angle of the neck 132 of the femoral stem component 130 and a length of the femoral stem component 130 and serve the notification to a display arranged proximal the patient. Therefore, in response to the force versus angular orientation curve and the target force versus angular orientation curve having a similarity score that exceeds a certain threshold, the controller 170 can generate a recommendation to maintain the current geometry of the femoral stem component 130, (e.g., due to a low risk of impingement, subluxation, and/or dislocation of the hip).

### 7.4 Surgeon Feedback

The controller 170 can then: generate a notification containing a recommendation identifying the second instrumented femoral head no, a particular shim geometry, or other adjustment to improve soft tissue tension balance on the hip over its range of motion; and serve this notification to the surgeon, such as via a display arranged in the operating theater.

In one implementation, the controller 170 can generate a notification containing the recommendation and interface with a display arranged in the operating theater to render the notification and the force versus angular orientation curve. Therefore, controller 170 can present both a recommendation for corrective action and data (i.e., the force versus angular orientation curve) supporting the recommendation (i.e., the data that informed the recommendation automatically generated by the controller 170.

### 7.5 Second Balancing Routine Prior To Acetabular Resection

The surgeon may then install the second instrumented femoral head no, install the particular shim, and/or execute the specified adjustment.

The surgeon then again moves the hip through its range of motion; and the reference module 150, the instrumented femoral head no, and the computing device cooperate to collect a next series of orientation and force data.

The controller 170 then implements methods and techniques described above to: generate a set of stress-position curves produced from these data; and recalculate a nominal artificial femoral head geometry and position based on these stress-position curves and a known geometry of the second instrumented femoral head 110; confirm the geometry and/or position of the second instrumented femoral head no on the femoral stem component 130; and/or identify and recommend a third instrumented femoral head no, shim geometry, or adjustment before the surgeon moves to a next step of the hip arthroplasty.

Then, in response to confirming the geometry and/or position of the second instrumented femoral head no on the femoral stem component 130, the controller 170 returns confirmation to ream the acetabulum to the surgeon.

For example, in one implementation, the controller 170 can, at a second time (e.g., during second balancing routine): access the fourth series of force data from a set of force sensors 112, the fifth series of orientation data from a set of inertial sensors 114, and the sixth series of reference orientation data from a reference module 150; calculate a second sequence of orientations of the instrumented femoral head 110 relative the pelvis based on the fifth series of orientation data and the sixth series of reference orientation data; and, based on the second sequence of orientations of the instrumented femoral head no and the fourth force data, calculate a second force versus angular orientation curve representing forces exerted on the instrumented femoral head no over a second range of motion of the femur. Therefore, the controller 170 can, during the second hip balancing routine, repeat the process of accessing the force data, the orientation data, and the reference orientation data, and generating the second force versus angular orientation curve. Then, the controller 170 can, based on certain features of the force versus angular orientation curve and/or based on the similarity of the force versus angular orientation curve to a target force versus angular orientation curve, determine to maintain or determine to adjust the geometry of the femoral stem component 130. For example, based on a similarity score for the second force versus angular orientation curve and a target force versus angular orientation curve exceeding a threshold score, the controller 170 can generate a recommendation to maintain the geometry of the femoral stem component 130.

### 7.6. Acetabular Component

The surgeon then: withdraws the femur from the pelvis to expose the acetabulum; reams the pelvis to receive an acetabular component; locates the acetabular component; and seats the instrumented femoral head no in the acetabular component. The surgeon then moves the hip through its range of motion while the reference module 150 and the instrumented femoral head 110 stream a next series of position data to the controller 170.

The controller 170 then implements methods and techniques described above to: compile orientations and force values received from the reference module 150 and the instrumented femoral head 110 into a surface and/or stress-position curves representing longitudinal force applied on the instrumented femoral head 110 as a function of flexion-extension, abduction-adduction, and/or internal-external motion of the hip.

The controller 170 can further implement methods and techniques described above to predict changes in depth position of the acetabular component that may yield: a target stress-position curve over a flexion-extension range of motion; a target stress-position curve over an abduction-adduction range of motion; a target stress-position curve over an internal-external range of motion; a target stress-position surface over complete three-dimensional range of motion; more than minimum force at any hip position within the range of motion; and/or less than a maximum force at any hip position within the range of motion.

Accordingly, the controller 170 can either recommend: replacement of the acetabular component with a second, thicker acetabular component; or further reaming of the acetabulum to deeper insertion of the acetabular component. For example, in response to detecting low force and nonlinear strain in and around the neutral hip position, the controller 170 can prompt the surgeon to replace the acetabular component with a second, thicker (or “deeper”) acetabular component. Conversely, in response to detecting high force and inelastic strain in and around the neutral hip position, the controller 170 can prompt the surgeon to further ream the acetabulum to accept further insertion of the acetabular component.

### 7.7. Acetabular Component Liner

Additionally, or alternatively, the surgeon may withdraw the femur from the pelvis to expose the acetabulum; ream the pelvis to receive an acetabular component; locate the acetabular component; install a liner (e.g., lipped liner, constrained liner) the acetabular component; and seat the instrumented femoral head 110 in the liner. The surgeon may then move the hip through its range of motion while the reference module 150 and the instrumented femoral head no stream a next series of position data to the controller 170.

The controller 170 then implements methods and techniques described above to: compile orientations and force values received from the reference module 150 and the instrumented femoral head 110 into a surface and/or stress-position curves representing longitudinal force applied on the instrumented femoral head 110 as a function of flexion-extension, abduction-adduction, and/or internal-external motion of the hip.

The controller 170 can further implement methods and techniques described above to predict changes in the liner that may yield: a target stress-position curve over a flexion-extension range of motion; a target stress-position curve over an abduction-adduction range of motion; a target stress-position curve over an internal-external rotation range of motion; a target stress-position surface over complete three-dimensional range of motion; more than minimum force at any hip position within the range of motion; and/or less than a maximum force at any hip position within the range of motion. Accordingly, the controller 170 can recommend, for example: replacement of the liner with a second, thicker liner; or replacement of the liner with a third, thinner liner. For example, in response to detecting a force on the instrumented femoral head 110 that falls below a threshold minimum force, the controller 170 can prompt the surgeon to replace the liner with a second, thicker liner.

### 7.8 Third Balancing Routine Prior to Acetabular Resection

The surgeon may then adjust the acetabular component accordingly and move the hip through its range of motion. The reference module 150, the instrumented femoral head no, and the computing device can cooperate to collect a next series of position data.

The controller 170 then implements methods and techniques described above to: generate a set of stress-position curves based on these orientation and force data; recalculate a nominal artificial femoral head geometry and position based on these stress-position curves and a known geometry of the second instrumented femoral head 110; and confirm the geometry and/or position of the acetabular component, the instrumented femoral head 110, and the femoral stem component 130 (or select and recommend other adjustments). Accordingly, in response to confirming the geometry and/or position of the second instrumented femoral head 110, the controller 170 can identify a particular artificial femoral head—in a kit of available artificial femoral heads—that best approximates the second (or last-selected) artificial femoral head geometry installed on the femoral stem component 130; and serve a recommendation specifying the particular artificial femoral head to the surgeon.

### 7.9 Surgery Completion

The surgeon may then install the particular artificial femoral head and close the patient to complete the surgery.

### 8. Lateral+Anteroposterior Force

In the foregoing implementation, the longitudinal force sensor 112 in the instrumented femoral head 110 is configured to output a signal representing force substantially parallel to the axis of the neck 132 of the femoral stem component 130. In the neutral position (or in another, specific position) of the hip, the force applied by the acetabulum or the acetabular component on the instrumented femoral head 110 may be substantially parallel to the axis of the neck 132 of the femoral stem component 130 and thus parallel to the sensible axis of the longitudinal force sensor 112. However, when the hip is manipulated outside of this neutral position, the force applied on the instrumented femoral head 110 by the acetabulum or the acetabular component on the instrumented femoral head 110 may be nonparallel to the sensible axis of the longitudinal force sensor 112 such that the longitudinal force sensor 112 detects a smaller component of this applied force at greater offsets from the neutral position. However, the component of this applied force detected by the longitudinal force sensor 112 may drop linearly with increasing offset positions of the hip from the neutral position. The controller 170 can thus: correlate nonlinear changes in longitudinal force detected by the longitudinal force sensor 112—concurrent with linear changes in the angular position of the instrumented femoral head 110 relative to the reference module 150—with nonlinear changes in tension on soft tissues around the hip; and then implement methods and techniques described above to interpret insufficient tension or inelastic deformation of these soft tissues and estimate changes in the position or geometry of the instrumented femoral head 110 to reduce such imbalances.

Alternatively, in one variation, the instrumented femoral head no further includes lateral and anteroposterior force sensors 112 configured to detect magnitude and direction) of forces in the lateral and anteroposterior directions, respectively, on the instrumented femoral head no.

In one implementation, the instrumented femoral head no further includes a two-axis linear stage: interposed between the shank 120 and the spherical shell 140; and configured to enable the spherical shell 140 to translate relative to the shank 120 along two axes perpendicular to the longitudinal axis of the neck 132 of the femoral stem component 130. In this implementation, the instrumented femoral head 110 can further include: a spring that centers the spherical shell 140 on the base; a lateral force sensor 112 configured to output a signal representing force on the spherical shell 140 in the lateral direction; and an anteroposterior force sensor 112 configured to output a signal representing force on the spherical shell 140 in the anteroposterior direction.

In this variation, the instrumented femoral head 110 can stream longitudinal, lateral, and anteroposterior force values to the controller 170 during hip balancing routines.

Accordingly, the controller 170 can combine concurrent orthogonal longitudinal, lateral, and anteroposterior force values received from the instrumented femoral head 110 to calculate total force applied to the instrumented femoral head 110. The controller 170 can then implement methods and techniques described above to: correlate nonlinear changes in total force on the instrumented femoral head 110—concurrent with linear changes in the angular position of the instrumented femoral head 110 relative to the reference module 150—with nonlinear changes in tension on soft tissues around the hip; interpret insufficient tension or inelastic deformation of these soft tissues; and estimate changes in the position or geometry of the instrumented femoral head 110 to reduce such imbalances.

### 9. Adjustable Femoral Stem Component

In one variation, the system includes an adjustable femoral stem component 130 including: a stem configured to insert into a femur; and a neck 132 pivotably coupled to the stem and configured to extend axially from the stem. For example, the neck 132: can be coupled to the stem via a two-axis, worm-driven gimbal; and can include proximal and distal ends coupled by an adjustable turnbuckle. A surgeon may thus temporarily install the adjustable femoral stem component 130 in a patient, set a target neck 132 geometry, and then finally install a (permanent) fixed femoral stem component 130 that best approximates this target neck 132 geometry set by the surgeon for the patient.

In this variation, the controller 170 can implement methods and techniques described above to: estimate changes in the angular position and length of the neck 132 of the femoral stem component 130; and then prompt the user to implement these changes by repositioning the adjustable femoral stem component 130.

In one implementation, the shank 120 of the instrumented femoral head 110 defines a bore configured to receive the proximal end of the neck 132 of the femoral stem component 130, and includes a depth micrometer including a spindle configured to locate the spherical shell 140 on the proximal end of a neck 132 of the femoral stem component 130 over a range of lengths, and configured to indicate a length, in the range of lengths, of the shank 120. Therefore, the instrumented femoral head no can include a bore, which facilitates attachment of the neck 132 of the femoral stem component 130 to the instrumented femoral head 110. Furthermore, the instrumented femoral head no can include a depth micrometer configured to indicate the length of the of the shank 120.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A system comprising:
an instrumented femoral head comprising:
a shank configured to seat on a proximal end of a neck of a femoral stem component installed on a proximal end of a femur;
a spherical shell arranged over the shank;
a set of force sensors configured to output:
a first series of force data representing forces acting on the spherical shell; and
a set of inertial sensors configured to output:
a second series of orientation data representing orientations of the instrumented femoral head;
a reference module configured to:
couple to a pelvis of a patient; and
output a third series of reference orientation data representing orientations of the pelvis; and
a controller configured to:
access the first series of force data, the second series of orientation data, and the third series of reference orientation data;
calculate a first sequence of orientations of the instrumented femoral head relative the pelvis based on the second series of orientation data and the third series of reference orientation data; and
based on the first sequence of orientations of the instrumented femoral head and the first series of force data, calculate a first force versus angular orientation curve representing forces exerted on the instrumented femoral head by an acetabulum in the pelvis over a first range of motion of the femur.

2. The system of claim 1:
wherein the instrumented femoral head further comprises a communication module:
arranged in the shank; and
configured to transmit the first series of force data, the second series of orientation data, and the third series of reference orientation data to the controller;
wherein the shank defines an external support surface;
wherein the spherical shell defines an internal contact surface facing the external contract surface of the shank; and
wherein the set of force sensors:
are interposed between the external contract surface of the shank and the internal contract surface of the spherical shell;
locate the spherical shell over the shank; and
communicate forces exerted on the instrumented femoral head by the acetabulum from the spherical shell onto the shank.

3. The system of claim 2:
wherein the external support surface of the shank comprises a base and a side; and
wherein the set of force sensors comprises:
a first force sensor arranged on the base of the external support surface shank;
a second force sensor arranged on the side of the external support surface and proximal a longitudinal axis of the shank;
a third force sensor arranged on the side of the external support surface opposite the second force sensor and proximal the longitudinal axis of the shank;
a fourth force sensor arranged on the side of the external support surface and proximal a lateral axis of the shank; and
a fifth force sensor arranged on the side of the external support surface opposite the fourth force sensor and proximal the lateral axis of the shank.

4. The system of claim 2:
wherein the external support surface of the shank comprises a semi-spherical external support surface; and
wherein the set of force sensors comprises a first force sensor, a second force sensor, and a third force sensor arranged on the semi-spherical external support surface and angularly spaced about a parallel of the semi-spherical external support surface, the parallel interposed between a proximal pole of the semi-spherical external support surface and an equator of the semi-spherical external support surface.

5. The system of claim 1:
wherein the set of inertial sensors of the instrumented femoral head comprises a first three-axis inertial measurement unit;
wherein the set of force sensors comprises a set of piezoelectric load cells supporting the spherical shell on the shank; and
wherein the reference module comprises a second three-axis inertial measurement unit.

6. The system of claim 1, wherein the instrumented femoral head:
further comprises a transmitter; and
is further configured to:
at a first time, identify a first temporal pattern of force fluctuations in the first series of force data, the first temporal pattern comprising a set of force peaks within a force activation range and occurring within an activation time interval;
in response to detecting the first temporal pattern:
activate the transmitter; and
trigger transmission of the first series of force data and the second series of orientation data, via the transmitter, to the controller;
at a second time, identify a second temporal pattern of force fluctuations in the first series of force data, the second pattern different from the first temporal pattern; and
in response to detecting the second temporal pattern, cease transmission of the first series of force data and the second series of orientation data to the controller.

7. The system of claim 1, wherein the instrumented femoral head:
further comprises a transmitter and a receiver; and
is further configured to:
at a first time, receive an activation signal from the controller via the receiver;
in response to receiving the activation signal, trigger transmission of the first series of force data and the second series of orientation data to the controller via the transmitter;
at a second time, receive a deactivation signal from the controller via the receiver; and
in response to receiving the deactivation signal, cease transmission of the first series of force data and the second series of orientation data.

8. The system of claim 1, wherein the controller is coupled to a display and is further configured to:
detect a global minimum force of the first force versus angular orientation curve; and
in response to the global minimum force exceeding a minimum force threshold:
detect impingement of the instrumented femoral head and the acetabulum resulting from a current length of the neck of the femoral stem component exceeding a target length;
predict the target length of the neck of the femoral stem component, the target length less than the current length of the neck of the femoral stem component, based on a difference between the global minimum force and the minimum force threshold;
generate a recommendation to shorten the current length of the neck of the femoral stem component according to the target length; and
transmit the recommendation to the display for presentation to a surgeon.

9. The system of claim 1, wherein the controller is further configured to:
detect a global minimum force of the first force versus angular orientation curve; and
in response to the global minimum force falling below a minimum force threshold:
detect subluxation of the femoral stem component in the acetabulum of the pelvis;
generate a recommendation to increase a length of the neck of the femoral stem component; and
render the recommendation on a display arranged proximal the patient.

10. The system of claim 1, wherein the controller is further configured to:
define the first range of motion of the femur comprising a set of angular orientations accessible to the femur, each angular orientation in the set of angular orientations associated with a force less than a maximum threshold force;
identify a first characteristic of the first force versus angular orientation curve, the first characteristic comprising a discontinuity in the force versus angular orientation curve, the discontinuity occurring within the first range of motion of the femur;
identify a second characteristic of the first force versus angular orientation curve, the second characteristic comprising a maximum force at the discontinuity, the maximum force at the discontinuity falling below the maximum threshold force;
match the first characteristic and the second characteristic to a template curve characteristic stored in a library and associated with a dislocation event; and
in response to matching the first characteristic and the second characteristic to the template curve characteristic:
predict dislocation of the femoral stem component from the acetabulum;
generate a notification indicating predicted dislocation of the femoral stem component from the acetabulum; and
render the notification on a display proximal the patient.

11. The system of claim 1, wherein the controller is further configured to:
define the first range of motion of the femur comprising a set of angular orientations accessible to the femur, each angular orientation in the set of angular orientations associated with a force below a maximum threshold force;
identify a first characteristic of the first force versus angular orientation curve, the first characteristic comprising a peak in the force versus angular orientation curve;
identify a second characteristic of the first force versus angular orientation curve, the second characteristic comprising a maximum force associated with the peak, the maximum force falling below the maximum threshold force;
match the first characteristic and the second characteristic to template curve characteristic stored in a library and associated with soft tissue impingement; and
in response to matching the first characteristic and the second characteristic to the template curve characteristic:
predict soft tissue impingement by the femoral stem component;
generate a notification indicating predicted soft tissue impingement; and
render the notification on a display proximal the patient.

12. The system of claim 1, wherein the controller is further configured to:
detect a first characteristic of the first force versus angular orientation curve, the first characteristic comprising an asymmetric profile of the first force versus angular orientation curve across the first range of motion;
detect impingement of the instrumented femoral head on the acetabulum based on the asymmetric profile of the first force versus angular orientation curve;
associate impingement of the instrumented femoral head on the acetabulum with an angle of the neck of the femoral stem component differing from a target angle of the femoral stem component;
calculate the target angle of the neck of the femoral stem component predicted to reduce asymmetry of the first force versus angular orientation curve;
generate a recommendation to adjust the angle of the neck of the femoral stem component to the target angle; and
render the recommendation on a display arranged proximal the patient.

13. The system of claim 1, wherein the controller is further configured to:
access a target force versus angular orientation curve;
calculate a similarity score for the first force versus angular orientation curve and the target force versus angular orientation curve; and
based on the similarity score exceeding a threshold similarity score:
generate a notification confirming an angle of the neck of the femoral stem component and a length of the femoral stem component; and
serve the notification to a display arranged proximal the patient.

14. The system of claim 1, wherein the controller is configured to:
access the first sequence of orientations of the instrumented femoral head, each orientation in the first sequence orientations comprising:
a first vector component indicating orientation of the instrumented femoral head parallel to a gravitational force;
a second vector component indicating orientation of the instrumented femoral head orthogonal to the first vector component; and
a third vector component indicating orientation of the instrumented femoral head orthogonal to the first vector component and the second vector component;
identify a first subset of orientations, in the first sequence of orientations, comprising second vector components within a narrow value range and first vector components and third vector components spanning a wide value range;
isolate a first subset of forces in the first series of force data corresponding to the first subset of orientations;
associate the first subset of orientations with the first range of motion for internal rotation and external rotation of the hip; and
generate the first force versus angular orientation curve comprising a force versus internal rotation angle and external rotation angle curve based on the first subset of forces and the first subset of orientations.

15. The system of claim 14, wherein the controller is further configured to:
identify a second subset of orientations, in the first sequence of orientations, comprising first vector components within the narrow value range and second vector components and third vector components spanning the wide value range;
identify a second subset of force data in the first series of force data corresponding to the second subset of orientations;
isolate a second subset of forces in the first series of force data corresponding to the second subset of orientations;
associate the second subset of orientations with a second range of motion for abduction and adduction of the hip; and
generate a force versus abduction and adduction curve based on the second subset of forces and the second subset of orientations.

16. An instrumented femoral head comprising:
a shank:
configured to seat on a proximal end of a neck of a femoral stem component installed on a proximal end of a femur; and
defining an external support surface;
a spherical shell:
arranged over the shank; and
defining an internal contact surface facing the external support surface of the shank;
a set of force sensors:
interposed between the external contract surface of the shank and the internal contract surface of the spherical shell; and
configured to:
locate the spherical shell over the shank;
communicate forces from the spherical shell into the shank; and
output a first series of force data representing forces acting on the spherical shell;
a set of inertial sensors:
arranged in the shank; and
configured to output a second series of orientation data representing orientations of the instrumented femoral head; and
a communication module:
arranged in the shank; and
configured to transmit the first series of force data, the second series of orientation data, and the third series of reference orientation data to a controller.

17. The instrumented femoral head of claim 16, wherein the shank:
further defines a bore configured to receive the proximal end of the neck of the femoral stem component; and
further comprises a depth micrometer:
comprising a spindle configured to locate the spherical shell on the proximal end of the neck of the femoral stem component over a range of lengths; and
configured to indicate a length, in the range of lengths, of the shank.

18. A method comprising:
at a first time:
accessing a first series of force data from a set of force sensors, a second series of orientation data from a set of inertial sensors, and the third series of reference orientation data from a reference module, the first series of force data representing forces acting on an instrumented femoral head installed on a proximal end of a femoral stem component attached to a femur of a patient, the second series of orientation data representing orientations of the instrumented femoral head, and the third series of reference orientation representing reference orientations of a pelvis of the patient;
calculating a first sequence of orientations of the instrumented femoral head relative the pelvis based on the second series of orientation data and the third series of reference orientation data;
based on the first sequence of orientations of the instrumented femoral head and the first series of force data, calculating a first force versus angular orientation curve representing forces exerted on the instrumented femoral head by an acetabulum in the pelvis over a first range of motion of the femur;
based on the first force versus angular orientation curve, identifying an impingement event, the impingement event representing impingement of the instrumented femoral head by an acetabulum or by a soft tissue;
predicting a target geometry of the femoral stem component associated with a target force versus angular orientation curve, the target force versus angular orientation curve representing forces exerted on the instrumented femoral head by an acetabulum in the pelvis over the first range of motion of the femur without impingement; and
generating a recommendation to adjust geometry of the femoral stem component to match the target geometry, the geometry of the femoral stem component defining an angle of the neck of the femoral stem component and a length of the neck of the femoral stem component.

19. The method of claim 18:
wherein accessing the first series of force data from a set of force sensors, the second series of orientation data from the set of inertial sensors, and the third series of reference orientation data from a reference module comprises:
access the first series of force data from a set of force sensors, the second series of orientation data from the set of inertial sensors, and the third series of reference orientation data from a reference module during flexion and extension motion of a hip of the patient;
wherein calculate a first force versus angular orientation curve representing forces exerted on the instrumented femoral head by an acetabulum in the pelvis over the first range of motion of the femur comprises:
calculating a first force versus angular orientation curve representing forces exerted on the instrumented femoral head by the acetabulum over a flexion-extension range of motion of the femur; and
further comprising:
access a fourth series of force data from a set of force sensors, a fifth series of orientation data from the set of inertial sensors, and a sixth series of reference orientation data from a reference module during abduction and adduction motion of the hip of the patient;
calculating a second sequence of orientations of the instrumented femoral head relative the pelvis based on the fifth series of orientation data and the sixth series of reference orientation data; and
based on the second sequence of orientations of the instrumented femoral head and the fourth series of force data, calculating a second force versus angular orientation curve representing forces exerted on the instrumented femoral head by an acetabulum in the pelvis over an abduction angle and adduction range of motion of the femur.

20. The method of claim 18, further comprising:
at a second time:
accessing a fourth series of force data from a set of force sensors, a fifth series of orientation data from the set of inertial sensors, and a sixth series of reference orientation data from a reference module;
calculating a second sequence of orientations of the instrumented femoral head relative the pelvis based on the fifth series of orientation data and the sixth series of reference orientation data;
based on the second sequence of orientations of the instrumented femoral head and the fourth series of force data, calculating a second force versus angular orientation curve representing forces exerted on the instrumented femoral head over a second range of motion of the femur; and
based on a similarity score for the second force versus angular orientation curve and a target force versus angular orientation curve exceeding a threshold score, generating a recommendation to maintain the geometry of the femoral stem component.
